(19) United States
(12) Patent Application Publication
(10) Pub. No.: US 2023/0226288 A1
(43) Pub. Date: Jul. 20, 2023

(54) IDENTIFICATION COMPONENT FOR AN AEROSOL-GENERATING DEVICE

(71) Applicant: Philip Morris Products S.A., Neuchâtel (CH)

(72) Inventors: Sze Chiek AW, Singapore (SG); Matteo BOLOGNA, Lausanne (CH); Teck Yan CHAN, Singapore (SG); Jean-Luc FRINGELI, Singapore (SG); Dani RUSCIO, Cressier (CH); Valerio OLIANA, Pully (CH); Richard MILHE, Divonne-les-Bains (FR); Alex FARROW, Pleasant Hill, CA (US)

(73) Assignee: Philip Morris Products S.A., Neuchâtel (CH)

(21) Appl. No.: 18/002,417

(22) PCT Filed: Jun. 22, 2021
(86) PCT No.: PCT/EP2021/067034

§ 371 Date: Dec. 19, 2022

(30) Foreign Application Priority Data
Jun. 25, 2020 (EP) 20182253.3

(51) Int. Cl. A61M 15/06; A61M 11/04

(52) U.S. Cl. CPC A61M 15/06; A61M 11/042; A61M 2205/6063

(57) ABSTRACT

An identification component for an aerosol-generating device is provided, the identification component including: a ring shaped body; a customizable surface; and connector for reversibly connecting the identification component to the aerosol-generating device, in which the identification component is a ring-shaped component that is configured to be mounted at an open end of a heating chamber of the aerosol-generating device. An aerosol-generating device including a reversibly connectable identification component is also provided. A system including the aerosol-generating device and a removal tool is also provided.

The present invention relates to an identification component for an aerosol-generating device. The identification component comprises a ring shaped body, a customizable surface, and connection means for reversibly connecting the identification component to the aerosol-generating device.

Nowadays consumers possess a plurality of electronic and non-electronic devices. These devices may play an important role in a consumer's daily life. Since, a tendency has evolved that users wish to personalize the appearance of their devices.

Such customization has not only aesthetic character. Customization is also helpful to facilitate identification of a consumer's device and to allow a consumer to differentiate the device from the devices of other consumers. By providing a customized external look to a device, it is possible to identify such device easily and efficiently.

It would be desirable to provide a customizable identification component that is configured to be reversibly mounted to an aerosol-generating device.

It would be further desirable to provide a customizable identification component, in which the customization is achievable without involving substantial structural modifications by a user.

It would be further desirable to provide an aerosol-generating device having a customization capability which is easily and quickly reversible, such that the aerosol-generating device may be brought to an initial state without effort.

It would be further desirable to provide a system comprising an aerosol-generating device, a customizable identification component, and a removal tool, whereby the removal tool is integrated in a part or an accessory of the aerosol-generating device.

According to an embodiment of the present invention there is provided an identification component for an aerosol-generating device. The identification component comprises a ring shaped body and a customizable surface. The identification component further comprises connection means for reversibly connecting the identification component to the aerosol-generating device.

The customizable surface allows the user to adapt the aesthetic character of the aerosol-generating device. At the same time, the customizable surface allows the user to facilitate identification and to differentiate the aerosol-generating device from the devices of other consumers. Thus, the possibility to customize the aerosol-generating device may increase the user experience and may foster the users relation to the aerosol-generating device.

The identification component may be used with any kind of aerosol-generating device. As used herein, an ‘aerosol-generating device’ relates to a device that interacts with an aerosol-forming substrate to generate an aerosol. The aerosol-forming substrate may be part of an aerosol-generating article. An aerosol-generating device may be a device that interacts with an aerosol-forming substrate of an aerosol-generating article. An aerosol-generating device may be a holder. The aerosol-generating device may be an electrically heated aerosol-generating device. The aerosol-generating device may comprise a housing, electric circuitry, a power supply, a heating chamber and a heating element. The housing may define a cavity that comprises the heating chamber and the heating element. The heating element may be part of the aerosol-generating article.

The aerosol-generating device is preferably a portable or handheld device that is comfortable to hold between the fingers of a single hand. The device may be substantially cylindrical in shape and may have a length of between 30 and 150 millimetres. The maximum diameter of the device is preferably between 5 and 30 millimetres.

The housing of the aerosol-generating device may be elongate. The housing may comprise any suitable material or combination of materials. Examples of suitable materials include metals, alloys, plastics or composite materials containing one or more of those materials, or thermoplastics that are suitable for food or pharmaceutical applications, for example polypropylene, polyetheretherketone (PEEK) and polyethylene. Preferably, the material is light and non-brittle.

The housing may comprise a mouthpiece. The housing may comprise at least one air inlet. The housing may comprise more than one air inlet. The mouthpiece may comprise at least one air inlet and at least one air outlet. The mouthpiece may comprise more than one air inlet. One or more of the air inlets may reduce the temperature of the aerosol before it is delivered to a user and may reduce the concentration of the aerosol before it is delivered to a user.

As used herein, the term ‘mouthpiece’ refers to a portion of an aerosol-generating device that during a puff is closest to the user's mouth.

The aerosol-generating device may include a sensor to activate the aerosol-generating device. The aerosol-generating device may include a user interface to activate the aerosol-generating device, for example a button to initiate heating of the aerosol-generating device or display to indicate a state of the aerosol-generating device or of the aerosol-forming substrate.

As used herein, the term ‘user experience’ refers to the act of consuming an aerosol-generating article. A user experience may include one or more consecutive puffs. The user experience also includes the time period in between puffs.

The customizable surface of the identification component may have a personalized appearance. The customizable surface of the identification component may have an individual colouring, engraving, pattern, or coating or may be customizable in any other way. Ready to use identification components that are provided with customized surfaces may be made available to the users. In addition, identification components may be provided having a surface that may be customized by the users themselves or that are customized based on the user's preferences.

The identification component may form a part of the housing of the aerosol-generating device. In aerosol-generating devices comprising a housing that defines a cavity with a heating chamber, the identification component may be used as a cover to be mounted at an end face of the housing. In aerosol-generating devices comprising a housing that defines a cavity with a heating chamber having an open end for insertion of an aerosol-generating article, the identification component may be used as a top ring that is to be mounted at an inlet portion of the heating chamber. The identification component may be a ring shaped component that is configured to be mounted at an open end of a heating chamber of an aerosol-generating device.

The end face of the housing is a very prominent portion of the aerosol generating device and therefore suitable for customization purposes. This part of the housing is clearly visible, in particular between puffs. In addition, the opening of the heating chamber of an aerosol-generating device points towards the user during the user experience. Thus, also for this reason this end face of the housing is a prominent part that is very often in the view field of the user and is therefore suitable to be used for customization purposes.

For mounting and removing of the identification component any kind of suitable connection means may be used. The connection means may be a screw connection, a form fitting connection, or a bayonet type connection. These kinds of connection means do not require application of increased force and can be readily utilised by users.

The connection means may be configured to produce haptic feedback, acoustic feedback or both haptic and acoustic feedback upon closure. In this way the connection means may provide a confirmation that a locked position of the identification component is reached. Such confirmation makes handling of the device easier and at the same time reassures the user of correct handling of the device.

The identification component may be easily removable, for example by hand. The identification component may also be removed with the help of a removal tool. If a removal tool is used, the identification component may further comprise coupling means configured to cooperate with such removal tool for mounting and removing the identification component from the aerosol-generating device.

The coupling means may be configured such as to allow the removal tool to transmit torque onto the identification component. For this purpose, the coupling means may include a recess with a non-circular profile. This recess having a non-circular profile may be configured to cooperate with a corresponding profile of the removal tool. In particular, if the identification component is a top ring of a housing of an aerosol-generating device, the through-hole defined by this top ring may be configured to have a non-circular profile. Preferably, such non-circular profile defines an opening that is large enough to allow insertion of an aerosol-generating article.

In principle any non-circular profile may be used for the coupling means. A symmetric (rotational symmetric or mirror symmetric) profile may be preferable, for example to simplify the alignment between the tool and the identification component for mounting or removing the identification component. A pentagonal or hexagonal profile may be particularly suitable. The profile may be polygonal. The corners of the polygonal profile may be rounded. The non-circular profile may be a 3-lobed, a 4-lobed or a 5-lobed profile. These profiles may define a sufficiently large opening to allow insertion of an aerosol-generating article and at the same time allow engagement of a removal tool to facilitate removal and connection of the identification component.

In embodiments in which the identification component forms a part of the housing of the aerosol-generating device, the identification component may include a sealing member to establish a sealing connection of the identification component to the housing. The sealing member may be a resilient sealing member. The sealing member may be a sealing ring. The sealing ring may be provided between the housing and the identification component. The sealing ring may be provided in a circumferential recess of the identification component, which recess is provided adjacent to the contact area between the identification component and the housing. In its uncompressed state the resilient sealing ring may have a dimension that is slightly larger than the circumferential recess, in which the sealing ring is located. Upon connection of the identification component to the housing the sealing ring is compressed and sealingly fills the gap between the identification component and the housing.

The identification component may further comprise attachment means. Such attachment means may be useful to temporarily fix the aerosol-generating device comprising the identification component to any suitable support. For example, the attachment means may be used to attach the aerosol-generating device to a part of a user's clothing. For this purpose the identification component may be configured as a pen clip.

The identification component may be configured to be flush with outer surface when not used for connection. Such design may be advantageous in particular when the aerosol-generating device is to be inserted into a charger. In this case the attachment means does not interfere with insertion of the aerosol-generating device into the charger. Such design may also be advantageous when the aerosol-generating device may be stored in a user's pocket between user experiences.

The identification component may also comprise an attachment means with a loop. The lop may be used to attach a lanyard so as to be able to carry the aerosol-generating device around a user's neck.

The loop of an attachment means may also be used to connect a personal identification item thereon. Such personal identification item may be a lanyard as such, but may also be a lucky charm or a tag.

The body of the identification component may have an outer surface with a non-circular cross-section. The body of the identification component may have an outer surface with an oval, polygonal, 5-lobed or hexagonal cross-section. The body of the identification component may have an outer surface with a cross-section having a shape that corresponds to the symmetry of the profile of the recess of the coupling means of the identification component.

Providing a identification component having a non-circular body may be convenient when the housing of the aerosol-generating device otherwise has a circular cross-section. In such case, the non-circular shape of the body of the identification component may prevent the aerosol-generating device from rolling down when positioned on not fully planar surfaces.

Preferably at least portions of the outer surface of the body of the identification component extend over the outer periphery of the housing of the aerosol-generating device to which the identification component is to be mounted. With such configuration, unintentional rotational movement of an aerosol-generating device may be most efficiently prevented.

The present invention is also directed to an aerosol-generating device comprising a reversibly connectable identification component as described herein. The aerosol-generating device may comprise a connection portion configured to co-operate with the connection means of the identification component. If the identification component has a screw connection portion, the aerosol-generating device is provided with a corresponding screw connection. If a haptic feedback, acoustic feedback or haptic and acoustic feedback upon closure of the identification component is to be generated, the connection portion of the aerosol-generating device may comprise structural elements that cooperate with corresponding structures of the identification component to generate such feedback.

The aerosol-generating device may comprise a housing defining a cavity extending from a proximal end of the housing. One or more air inlets may be formed at the proximal end of the housing. The identification component may be configured to be mounted at the proximal end of the housing, and may be configured to adjust the flow cross-sectional area of the one or more air inlets formed at the proximal end of the housing. Such design may allow a user to adjust the air flow according to the personal needs and preferences. Thereby the user experience may be enhanced.

The identification component may comprise two ring-shaped portions, wherein at least one of the ring-shaped portions is rotatably mounted with respect to the other ring-shaped portion. Thus, the two ring-shaped portions may be rotationally mounted with respect to each other.

The flow cross-sectional area of the one or more air inlets may be adjusted by rotation of the rotatably mounted ring-shaped portion. The two portions may be coaxially arranged. The two portions may each define a through-hole having a non-circular cross-section.

The two portions may each define a through-hole having an identical non-circular cross-section. The two portions may be rotated such that they are in an identical orientation. The flow cross-sectional area of the one or more air inlets may be maximized when the two ring-shaped portions of the identification component are in an identical orientation. The two portions may also be rotated such that they are in an offset relative orientation. The flow cross-sectional area of the one or more air inlets may be minimized when the two ring-shaped portions of the identification component are rotationally offset from each other.

The two portions may be rotated such as to resume any intermediate orientation. By choosing any intermediate rotational position, the flow cross-sectional area of the one or more air inlets may be adjusted to a specific user preference.

A first ring-shaped portion of the identification component may be configured to comprise the connection means for connecting the identification component to the aerosol-generating device. A second ring-shaped portion of the identification component may be rotatably mounted on the first ring-shaped portion. By rotating the second ring-shaped portion with respect to the first ring-shaped portion, adjustment of the cross-sectional air flow passage may be adjusted.

A rotation mechanism may be provided in order to allow to adjust the rotation angle between the two rotatably mounted portions of the identification component. Such rotation mechanism may ensure that the adjustment of the rotation angle between the two ring-shaped portions of the identification component may be reliably and repeatedly adjusted.

The present invention is also directed to a system comprising an aerosol-generating device with a reversibly connectable identification component as described herein, and a removal tool for mounting and removing the identification component from the aerosol-generating device.

The removal tool may comprise a structure having a profile that, at least partially, corresponds to the non-circular profile of a recess of coupling means of the reversibly connectable identification component.

The removal tool may be formed as a male or protruding structure that can be inserted into the recess of the coupling means of the reversibly connectable identification component.

The removal tool may be part of or fixed to an accessory of the aerosol-generating system.

The aerosol-generating system may comprise an accessory in the form of a cap for closing the open end of the cavity of the housing of the aerosol-generating device. The cap may have an outer shape that corresponds to the outer shape of the housing of the aerosol-generating device. The removal tool may be provided as a male profile protruding from the inner surface of the cap. The inner surface of the cap is the surface that points towards the aerosol-generating device when the cap is mounted to the aerosol-generating device. If the housing of the aerosol-generating device has a circular outer shape, the cap may have a corresponding circular shape, and the male profile may protrude from the circular inner surface of the cap.

For removing the identification element from the housing of the aerosol-generating device, the cap is mounted to the aerosol generating device. For this purpose the protruding male profile of the cap is inserted into the recess of the coupling means of the identification element. The cap may then be rotated to transfer a torque onto the identification element, and to thereby remove the identification element from the housing of the aerosol-generating device. When the device is not in use, the cap may be at the same time used as a cover to protect the cavity of the housing of the aerosol-generating device from damage and intrusion of dust or debris.

The aerosol-generating device may also comprise further accessories such as a charging cable. The removal tool may be a part of or connected to the charging cable. The removal tool may be mounted on a strip that is connected to the charging cable. The removal tool may be part of a plug of the charging cable. Since the charging cable is typically a mandatory accessory of an electronic aerosol-generating device, provision of the removal tool as a part of such mandatory accessory does not increase the number of individual components of the aerosol-generating system. Thus, the user will have to carry less components for ensuring the full functionality of the aerosol-generating system.

The charging cable may also be provided with a component carrying a female coupling element. The female coupling element may be used to attach the cap thereto, when the cap is removed from the aerosol-generating device. In this way the cap may at any time be connected to a part of the aerosol-generating system. In this way the risk of losing the cap may be reduced.

Typically charging cables have a plug on either end of the charging cable. When one plug of the charging cable is provided with the removal tool, the other plug of the charging cable may be provided with a corresponding female coupling element. When the charging cable is not in use, the cable may be rolled up and the plug with the removal tool may be coupled to the plug with the female coupling element. By coupling the two plugs together, the charging cable may be kept in the rolled-up configuration when the charging cable is not used for charging.

An aerosol-generating system is a combination of an aerosol-generating device, one or more aerosol-generating articles for use with the aerosol-generating device, and one or more accessories. The aerosol-generating system may include additional components, such as, for example a charging unit for recharging an on-board electric power supply in an electrically operated or electric aerosol-generating device.

A user may purchase additional identification elements. The user may change identification elements depending on personal preferences. To hold the identification elements in place, the package may be provided with a tray that is provided with a male coupling element. In the package the identification element may be coupled to this male coupling element. The male coupling element may be configured removable from the tray, such that this male coupling element may subsequently also be used as a removal tool. For this purpose, the tray may be provided with scoring lines such that the male coupling element may be removed from the tray.

Below, there is provided a non-exhaustive list of non-limiting examples. Any one or more of the features of these examples may be combined with any one or more features of another example, embodiment, or aspect described herein.

Example 1: The identification component for an aerosol-generating device, the identification component comprising a ring shaped body, a customizable surface, and connection means for reversibly connecting the identification component to the aerosol-generating device.

Example 2: Identification component according to example 1, wherein the identification component forms a part of a housing of the aerosol-generating device.

Example 3: Identification component according to any preceding example, wherein the identification component is a top ring of the housing of the aerosol-generating device.

Example 4: Identification component according to any preceding example, wherein the customizable surface may comprise a personalized appearance comprising one or more of an individual colouring, engraving, pattern, or coating.

Example 5: Identification component according to any preceding example, wherein the connection means may be a screw connection, a form fitting connection, or a bayonet type connection.

Example 6: Identification component according to any preceding example, wherein the connection means may be configured to produce haptic feedback upon closure to confirm a locked position of the identification component.

Example 7: Identification component according to any preceding example, further comprising coupling means configured to cooperate with a removal tool for mounting and removing the identification component from the aerosol-generating device.

Example 8: Identification component according to example 7, wherein the coupling means are configured such as to allow the removal tool to transmit torque onto the identification component.

Example 9: Identification component according to example 7 or 8, wherein the coupling means include a recess with a non-circular profile configured to cooperate with a mating profile of the removal tool.

Example 10: Identification component according to example 9, wherein the non-circular profile is a polygonal profile, in particular a 5-lobed profile or a hexagonal profile

Example 11: Identification component according to any preceding example, wherein the identification component may be a ring shaped component that is configured to be mounted at an open end of a heating chamber of an aerosol-generating device.

Example 12: Identification component according to any preceding example, wherein the identification component may include a sealing member, in particular a resilient sealing member.

Example 13: Identification component according to any preceding example, further comprising attachment means.

Example 14: Identification component according to example 13, wherein the attachment means is configured to be flush with outer surface when not used for connection.

Example 15: Identification component according to example 13 or 14, wherein the attachment means is configured as a pen clip.

Example 16: Identification component according to any of examples 13 to 15, wherein the attachment means may comprise a loop.

Example 17: Identification component according to example 16, wherein the loop of the attachment means is configured to connect a personal identification item thereon.

Example 18: Identification component according to any of examples 16 or 17, wherein the personal identification item is a lanyard, a lucky charm, or a tag.

Example 19: Identification component according to any preceding example, wherein the body of the identification component has an outer surface with a non-circular cross-section.

Example 20: Identification component according to any preceding example, wherein the body of the identification component has an outer surface with an oval, polygonal, 5-lobed or hexagonal cross-section.

Example 21: Identification component according to any of examples 9 to 20, wherein the body of the identification component has an outer surface with a cross-section having a shape that corresponds to the symmetry of the profile of the recess of the coupling means of the identification component.

Example 22: Identification component according to any preceding example, wherein at least portions of the outer surface of the body of the identification component extend over the outer periphery of the housing of the aerosol-generating device to which the identification component is to be mounted.

Example 23: Aerosol-generating device comprising a reversibly connectable identification component according to any preceding example.

Example 24: Aerosol-generating device according to example 23, comprising a connection portion configured to co-operate with the connection means of the identification component.

Example 25: Aerosol-generating device according to any of examples 23 or 24, comprising a housing defining cavity extending from a proximal end of the housing, wherein the identification component is configured to be mounted at the proximal end of the housing, wherein or more air inlets are formed at the proximal end of the housing, and wherein the identification component is configured to adjust the flow cross-sectional area of the one or more air inlets.

Example 26: Aerosol-generating device according to example 25, wherein the identification component comprises two ring-shaped portions, wherein at least one of the ring-shaped portions is rotatably mounted with respect to the other ring-shaped portion.

Example 27: Aerosol-generating device according to example 26, wherein the flow cross-sectional area of the one or more air inlets is adjusted by rotation of the rotatably mounted ring-shaped portion.

Example 28: Aerosol-generating device according to any of examples 26 or 27, wherein the two portions are both coaxially arranged and each define a through-hole having a non-circular cross-section.

Example 29: Aerosol-generating device according to any of examples 26 to 28, wherein the flow cross-sectional area of the one or more air inlets is maximized when the two ring-shaped portions of the identification component are in identical orientation, and wherein the flow cross-sectional area of the one or more air inlets is minimized when the two ring-shaped portions of the identification component are rotationally offset from each other.

Example 30: Aerosol-generating device according to any of examples 26 to 29, wherein a first ring-shaped portion of the identification component is configured to comprise the connection means, and wherein a second ring-shaped portion of the identification component is rotatably mounted on the first ring-shaped portion.

Example 31: Aerosol-generating device according to any of examples 26 to 30, wherein a rotation mechanism allows to adjust the rotation angle between the two rotatably mounted portions of the identification component.

Example 32: System comprising an aerosol-generating device according to any of examples 23 to 31 with a reversibly connectable identification component according to any of examples 1 to 22 and a removal tool for mounting and removing the identification component from the aerosol-generating device.

Example 33: System according to example 32, wherein the removal tool comprises a structure having a profile that corresponds to the non-circular profile of the recess of the coupling means.

Example 34: System according to examples 32 or 33, wherein the removal tool is part of or fixed to another accessory of the aerosol-generating device/system.

Example 35: System according to any of examples 32 to 34, wherein the removal tool is included in a cap for closing/protecting the heating chamber.

Example 36: System according to any of examples 32 to 35, wherein the removal tool is attached to a charging cable for the aerosol-generating device.

Example 37: System according to example 36, wherein the removal tool is attached to a first connector of a charging cable for the aerosol-generating device, and wherein a corresponding structure is provided to the second connector of the charging cable, such that the removal tool may also be used for keeping the charging cable in a rolled up configuration.

Features described in relation to one embodiment may equally be applied to other embodiments of the invention.

The invention will be further described, by way of example only, with reference to the accompanying drawings in which:

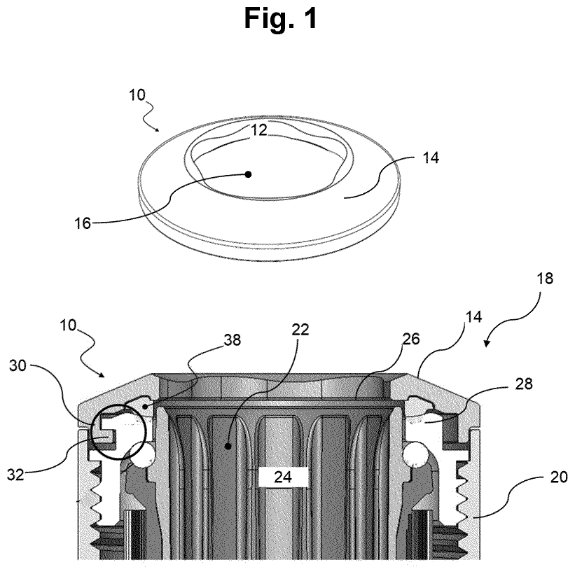

FIG. 1 shows a proximal end of an aerosol-generating device with identification component;

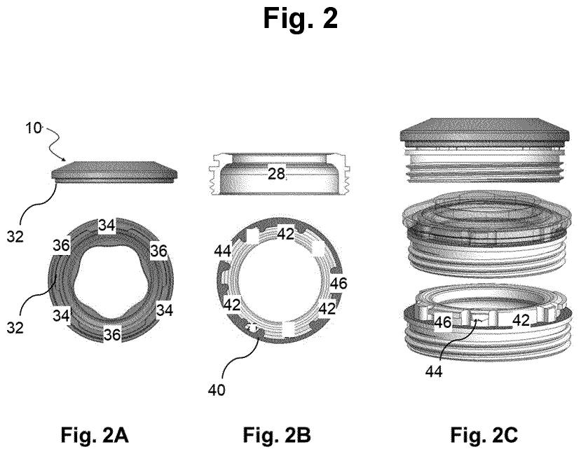

FIG. 2 shows an identification component with bayonet connection means;

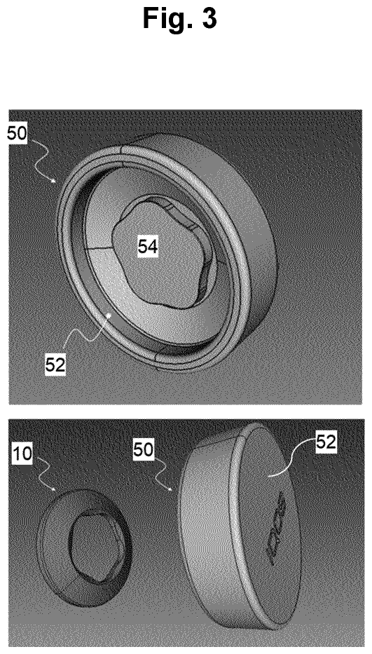

FIG. 3 shows a cap shaped removal tool for the identification component;

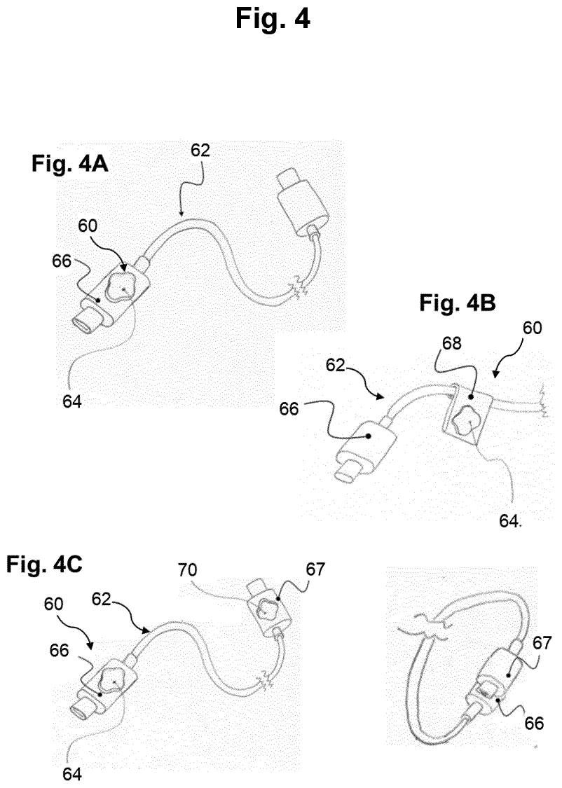

FIG. 4 shows further embodiments of removal tools for the identification component; and

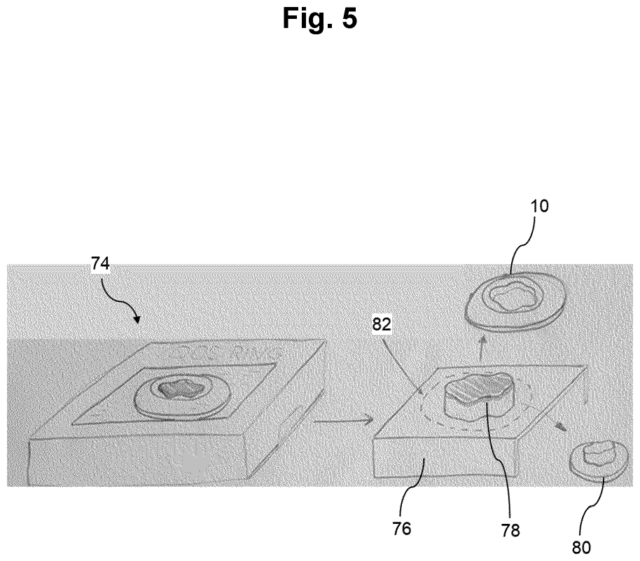

FIG. 5 shows a sales package for the identification component;

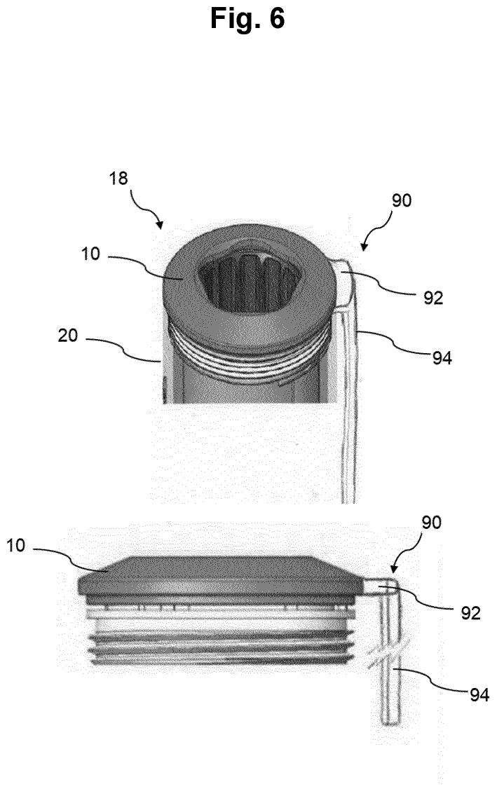

FIG. 6 shows an identification component with a first attachment means;

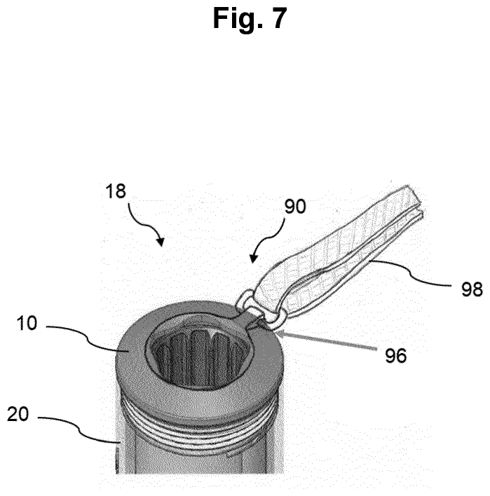

FIG. 7 shows an identification component with a second attachment means;

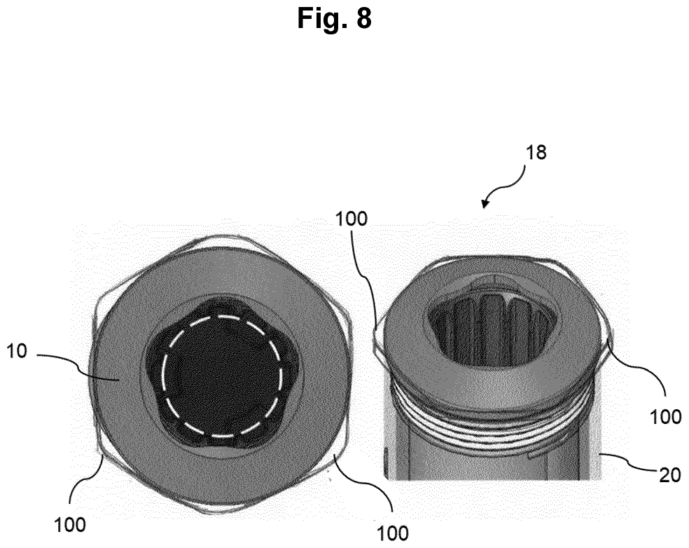

FIG. 8 shows an identification component with a non-circular outer cross-section;

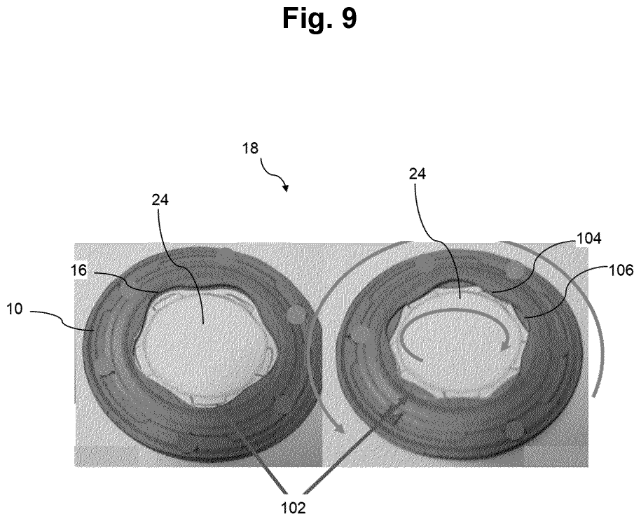

FIG. 9 shows an identification component with two ring-shaped portions;

FIG. 1 shows an embodiment of an identification component 10 for an aerosol-generating device 18. At the top of FIG. 1 a perspective view of the identification component 10 is provided. The identification component 10 has an essentially ring shaped body 12 and a customizable surface 14. The ring shaped body 12 defines a central through-hole 16 having a non-circular cross-section. Through-hole 16 has a five-lobed cross-section.

In the bottom view of FIG. 1 a cross-sectional view of the identification component 10, which is mounted to a proximal end of an aerosol-generating device 18, is depicted. The aerosol-generating device 18 comprises a housing 20 that defines a cavity 22 with a heating chamber 24. The cavity 22 has an open end 26 for insertion of an aerosol-generating article. The open end 26 comprises a connection portion 28 to which the identification component 10 can be mounted. In this embodiment the identification component 10 is used as a top ring that is to be mounted to the connection portion 28 at the open end 26 of the heating chamber 24 of the aerosol-generating device 18. In this embodiment the identification component 10 essentially forms a part of the housing of the aerosol-generating device 18. The identification component 10 forms the proximal end face of the elongate housing of the aerosol-generating device 18.

Connection means 30 are provided for reversibly mounting the identification component 10 to the aerosol-generating device 18. In this embodiment the connection means 30 is a bayonet connection. The bayonet connection is established between the connection portion 28 of the aerosol-generating device 18 and a connection portion 32 of the identification component 10. The identification component 10 can be easily mounted to the aerosol-generating device 18 by a relative rotation of only 120 degrees.

The identification component 10 comprises an internal sealing member 38. Sealing member 38 is provided as a ring shaped element that is located in a circumferential recess of the identification component 10. The sealing member 38 creates a tight coupling between the identification component and the open end 26 of the heating chamber 24. With the use of sealing member 38 leakage may be avoided. The sealing member 38 may be slightly compressed during mounting. This may provide the user with a better haptic sensation during mounting and removing of the identification component 10.

The connection means 30 are further illustrated with reference to FIG. 2. FIG. 2a shows a lateral view and a bottom-up perspective of the ring shaped identification component 10. In the bottom-up perspective of the ring shaped identification component 10 the connection portion 32 of the identification component 10 is visible. The connection portion 32 comprises three protrusions 34 and three recesses 36. The protrusions 34 and the recesses 36 are arranged in alternate sequence along the circumference of the connection portion 32 of the identification component 10.

FIG. 2B, similarly, shows a lateral view and a plan view of the connection portion 28 at the open end 26 of the heating chamber 24 of the aerosol-generating device 18. The connection portion 28 comprises a base 40 and three recessed elements 42. The recessed elements 42 are configured to allow the downward sliding of protrusions 34 of the identification element 10. The connection portion 28 further comprises portions 44 and portions 46 that are configured for locking the identification component 10 upon rotation.

FIG. 2C shows a perspective view that illustrates the operation principle of the bayonet connection means 30 between the identification component 10 and the connection portion 28 of the aerosol-generating device 18. As shown in the bottom part of FIG. 2C the identification component 10 is set onto the connection portion 28 in such way that the recessed elements 42 of the connection portion 28 are aligned with and receive the protrusions 34 of connection portion 32 of the identification component 10. Subsequently, the identification component 10 is rotated clockwise until protrusions 34 engage with portions 44 and portions 46 of connection portion 28. Portion 44 has a tapered end which facilitates the sliding of the protrusions 34 between the base 40 and the protruding portions 44 and 46.

When protrusions 34 are fully engaged with the protruding portions 46, the bayonet connection 30 between the the connection portion 28 of the aerosol-generating device 18 and the connection portion 32 of the identification component 10 is established.

As can be seen in the lower view of FIG. 2C protruding portions 44 extend a little further down than protruding portions 46. Thus, when protrusions 34 reach the protruding portions 46, the protrusions 34 snap upwards against protruding portions 46. This generates a slight click which serves as a haptic feedback, informing the user that the identification component 10 is in the locked position. This haptic feedback may be increased by the compressed sealing member 38 (not shown in FIG. 2). Moreover, since protruding portions 44 extend a little further down than protruding portions 46 unintentional return of the identification component 10 into the decoupled configuration is avoided.

For intentional removal of the identification component 10 a removal tool 50 may be used. An embodiment of a removal tool 50 is depicted in FIG. 3. This removal tool 50 is provided in the form of cap. The cap has a body 52 with an essentially cylindrical shape and a diameter that corresponds to the diameter of the housing of the aerosol-generating device 18.

At its internal portion the cap has protruding element 54 that has a shape that corresponds to the shape of the non-circular cross-section of the central through-hole 16 of the identification component 10.

For removing the identification component 10 from the aerosol-generating device 18, the cap is arranged on the identification component 10 in such way that the protruding or male element 54 is inserted into the central through-hole 16 of the identification component 10.

The cap is then rotated thereby transferring the rotational movement onto the identification component 10. The cap is rotated until the bayonet connection 30 between the identification component 10 and the aerosol-generating device 18 is decoupled. The identification component 10 may then be removed from the aerosol-generating device 18.

For mounting the identification component 10 to the aerosol-generating device 18, the process is essentially reversed. The cap is again arranged on the identification component 10. The identification component 10 is then arranged on the aerosol-generating device 18 and is rotated to establish the bayonet connection 30.

The cap has an overall diameter that corresponds to the diameter of the housing of the aerosol-generating device 18. The cap can be used to cover the heating chamber when the aerosol-generating device 18 is not used. In this way the heating chamber is conveniently protected from ingress of debris or obstacles that may damage the internal structure or components of the heating chamber 24.

In FIG. 4 further additional options for providing a removal tool 60 are depicted. In the embodiment of FIG. 4A the male component of the removal tool 60 is provided as a protruding element 64 on a first end plug 66 of a charging cable 62 of the aerosol-generating device 18. The charging cable 62 is a necessary accessory of an aerosol-generating system comprising an electrically operated aerosol-generating device 18. By providing the removal tool 60 as a part of such charging cable 62, the user will have to carry a lesser number of articles for ensuring the full functionality of the aerosol-generating system.

The removal tool 60 needs not necessarily to be provided at one of the plugs 66 of the charging cable 62 of the aerosol-generating system. Instead the protruding element 64 may also be provided on a strip 68 that is attached to the charging cable 62. Such embodiment is depicted in FIG. 4B. Also in this embodiment the risk of losing the removal tool 60 is considerably reduced.

As depicted in FIG. 4C, the charging cable may additionally comprise on the second plug 67 a female element 70, which has essentially the same shape as the non-circular recess 12 of the identification component 10 and is configured to mate with protruding element 64 of the first plug 66. Female element 70 and male element 64 may be configured to establish a pressure-fit type connection. When the removal tool 60 is not in use, the two plugs 66, 67 of the charging cable 62 may be connected to each other. This provides a quick and efficient way to maintain the charging cable 62 in a wound-up configuration.

It is envisaged that the user may purchase additional identification elements 10 to customize the aerosol-generating device 18. For this purpose the identification component 10 may be sold in a package 74 as depicted in FIG. 5. The identification component 10 may be placed in the package 74 within a single-line up package tray 76. The tray 76 may be used to maintain the identification component 10 at a desired position in the package 74. For this purpose the tray 76 may be provided with a protruding component 78 having a non-circular cross-section corresponding to the shape of the recess 12 of the identification component 10. The identification component 10 may be removed from the protruding component 78 and may be mounted to an aerosol-generating device 18.

The protruding component 78 may also be used as an additional removal tool 80. For this purpose, the tray 76 is provided with a pre-cut line 82 extending around the protruding component 78. The protruding component 78 including the tray portion within the pre-cut line 82 may be detached from the remainder of the tray 76. After detachment the protruding component 78 may be used as a mounting or removal tool 80 for the identification component 10. In this way the protruding component 78 may at the same time be used for maintain the identification component 10 in a desired position within the package 74, and as a mounting and removal tool 80 after purchase of the identification component 10.

FIG. 6 shows an identification component 10 comprising an attachment means 90 in the form of a pen clip. The pen clip comprises a base element 92 and an arm 94 that extends parallel to the housing 20 of the aerosol-generating device 18. With this attachment means 90 the aerosol-generating device 18 may be temporarily attached for example to a user's shirt pocket.

FIG. 7 shows an identification component 10 comprising an attachment means 90 comprising a loop 96. The loop 96 allows for attachment of a lanyard 98. The lanyard 98 may be used to further personalize the identification component 10 and the aerosol-generating device 18, respectively. The lanyard 98 may also be used to hang the aerosol-generating device 18 around a user's neck between or during user experiences.

FIG. 8 shows an identification component 10 having a non-circular outer cross-section. In FIG. 8 the identification component 10 has a hexagonal outer cross-section. The angles 100 of the hexagonal outer surface of the identification component 10 extend over the cylindrical outer surface of the housing 20 of the aerosol-generating device 18. In this way, the identification component 10 may help to prevent the aerosol-generating device 18 from rolling off of a non-planar surface.

FIG. 9 shows an enlarged view of an opening to a heating chamber 24 formed at a proximal end of an aerosol-generating device 18. The ring-shaped identification component 10 is mounted to the housing 20 (not shown) at the open end of the heating chamber 24. The heating chamber 24 is configured to receive a cylindrical aerosol-generating article. At the periphery of the heating chamber 24 a plurality of air inlets 102 are formed. The identification component 10 comprises two ring-shaped portions 104,106 each defining a central through-hole 16 having a non-circular cross-section, and which allow a user to adjust the air flow through the air inlets 102 of the aerosol-generating device 18. For this purpose the two ring-shaped portions 104,106 are configured rotatable with respect to each other. This is indicated in FIG. 9 by the circular arrows.

The left view in FIG. 9 shows the two ring-shaped portions 104,106 in a first configuration in which the two ring-shaped portions 104,106 are in an identical orientation. The ring-shaped portions 104,106 are rotated such that the air inlets 102 of the heating chamber 24 of the aerosol-generating device 18 are fully exposed. In this configuration the flow cross-sectional area of the air inlets 102 is maximized.

In the right view in FIG. 9 the two ring-shaped portions 104,106 are depicted in a second configuration, in which the two ring-shaped portions 104,106 are in a fully offset rotational orientation. The ring-shaped portions 104,106 are rotated such that the air inlets 102 of the heating chamber 24 of the aerosol-generating device 18 are partly covered. In this configuration the flow cross-sectional area of the air inlets 102 is minimized.

The degree of coverage of the air inlets 102 may be adjusted by the user by choosing any intermediate rotational orientation of the two ring shaped portions 104,106 of the identification component 10.

1.-36. (canceled)

37. An identification component for an aerosol-generating device, the identification component comprising:
a ring shaped body;
a customizable surface; and
connection means for reversibly connecting the identification component to the aerosol-generating device,
wherein the identification component is a ring-shaped component that is configured to be mounted at an open end of a heating chamber of the aerosol-generating device.

38. The identification component according to claim 37, wherein the identification component is a top ring of a housing of the aerosol-generating device.

39. The identification component according to claim 37, wherein the customizable surface comprises a personalized appearance comprising one or more of an individual coloring, engraving, pattern, or coating.

40. The identification component according to claim 37, wherein the connection means is a screw connection, a form fitting connection, or a bayonet type connection.

41. The identification component according to claim 37, wherein the connection means is configured to produce haptic feedback upon closure to confirm a locked position of the identification component.

42. The identification component according to claim 37, further comprising coupling means configured to cooperate with a removal tool for mounting and removing the identification component from the aerosol-generating device.

43. The identification component according to claim 42, wherein the coupling means are configured to allow the removal tool to transmit torque onto the identification component.

44. The identification component according to claim 42, wherein the coupling means include a recess with a non-circular profile configured to cooperate with a mating profile of the removal tool.

45. The identification component according to claim 37, wherein the identification component includes a sealing member.

46. The identification component according to claim 45, wherein the sealing member is a resilient sealing member.

47. An aerosol-generating device comprising a reversibly connectable identification component according to claim 37.

48. The aerosol-generating device according to claim 47, further comprising a connection portion configured to cooperate with the connection means of the identification component.

49. The aerosol-generating device according to claim 47,
further comprising a housing defining a cavity extending from a proximal end of the housing,
wherein the identification component is configured to be mounted at the proximal end of the housing,
wherein one or more air inlets are formed at the proximal end of the housing, and
wherein the identification component is further configured to adjust a flow cross-sectional area of the one or more air inlets.

50. The aerosol-generating device according to claim 47,
wherein the identification component comprises two ring-shaped portions,
wherein at least one of the ring-shaped portions is rotatably mounted with respect to the other ring-shaped portion, and
wherein the flow cross-sectional area of the one or more air inlets is adjusted by rotation of the rotatably mounted ring-shaped portion.

51. A system, comprising:
an aerosol-generating device comprising a reversibly connectable identification component according to claim 37; and
a removal tool configured to mount and to remove the identification component from the aerosol-generating device.

52. The system according to claim 51, wherein the removal tool comprises a structure having a profile that corresponds to a non-circular profile of the recess of the coupling means.
